(19) United States
(12) Patent Application Publication
(10) Pub. No.: US 2009/0299312 A1
(43) Pub. Date: Dec. 3, 2009

(54) Twisted, Compressed Substrates as Wetness Indicators in Absorbent Articles

(75) Inventors: John Gavin MacDonald, Decatur, GA (US); Jin Heo, Yongin-si (KR); Jaeho Kim, Roswell, GA (US)

Correspondence Address: DORITY & MANNING, P.A., POST OFFICE BOX 1449, GREENVILLE, SC, 29602-1449, US

(73) Assignee: Kimberly-Clark Worldwide, Inc., Neenah, WI (US)

(21) Appl. No.: 12/129,795

(22) Filed: May 30, 2008

(51) Int. Cl. A61F 13/42; D02J 13/00

(52) U.S. Cl. 604/361; 264/103

(57) ABSTRACT

Absorbent articles including a twisted, compressed substrate are generally described. The twisted, compressed substrate of the present invention is configured to expand toward the skin of the wearer (i.e., in the z-direction of the absorbent article perpendicular to the plane of the absorbent article) upon contact with a liquid. However, the expansion of the twisted, compressed substrate is substantially limited to the z-direction. Also, upon contact with a liquid, the twisted, compressed substrate unwinds to penetrate through the absorbent core of the absorbent article to reduce any pressure put on the absorbent core during expansion.

### BACKGROUND OF THE INVENTION

Expanding substrates that expand upon contact with a liquid are useful in many applications. For example, some absorbent articles intended for personal wear during toilet training include means for alerting a child to urination without leaving a substantial amount of wetness against the skin. One example of training pants intended to provide a sensory indication of urination includes an element that changes size after urination (e.g., expanding upon wetting). The expanding substrate can be used in the article to alert the wearer that an insult to the article has occurred.

However, due to the placement of the expanding substrate within the construction of the absorbent article, the expanding substrate presses against the other layers of the absorbent article upon wetting. This pressure on the other layers, particularly on the absorbent core, can impair the absorbent article's ability to capture and retain the bodily fluids insulting the article and causing the expanding substrate to expand. For example, the pressure resulting from the expanding substrate can cause the expanding substrate to press against the absorbent core.

While there has been progress in the design of expanding substrates, such as those capable of alerting a wearer to a release of liquid body exudates, there continues to be a need for an expanding substrate that avoids asserting undue pressure on the absorbent core of the absorbent article.

### SUMMARY OF THE INVENTION

Objects and advantages of the invention will be set forth in part in the following description, or may be obvious from the description, or may be learned through practice of the invention.

In one embodiment, the present invention is generally directed to an absorbent article configured to alert a wearer of an insult. The absorbent article includes an absorbent core positioned between a liquid-permeable layer and a liquid-impermeable layer, A twisted, compressed substrate is positioned between the liquid-permeable layer and the liquid-impermeable layer and is configured to expand in the z-direction upon contact with a liquid to form an expanded substrate without substantially expanding in either the x-direction or the y-direction. The twisted, compressed substrate is also configured to unwind upon contact with a liquid.

In another embodiment, the present invention is generally directed to a method of making a twisted, compressed substrate. A web material is twisted into a twisted, cylindrical tube and positioned into an elongated barrel. The twisted, cylindrical tube is subjected to a compression force in a direction of the elongation of the barrel. The compression force is provided by moving a pressing rod through the elongated barrel.

Other features and aspects of the present invention are discussed in greater detail below.

### BRIEF DESCRIPTION OF THE DRAWINGS

A full and enabling disclosure of the present invention, including the best mode thereof to one skilled in the art, is set forth more particularly in the remainder of the specification, which includes reference to the accompanying figures, in which:

FIG. 1A shows an exemplary twisted, compressed substrate in its compressed state;

FIG. 1B shows the exemplary twisted, compressed substrate of FIG. 1A in its expanded state;

FIGS. 2A and 2B show an exemplary absorbent article including a wetness indicator in both its compressed and expanded states, respectfully;

FIG. 3 shows the construction of an exemplary diaper including a wetness indicator according to one embodiment of the present invention;

FIG. 4 shows an exemplary training pant including a wetness indicator according to one embodiment of the present invention;

FIG. 5 shows an exemplary sanitary napkin for feminine care including a wetness indicator according to one embodiment of the present invention; and

FIGS. 6 and 7 show an exemplary process of rolling and twisting a nonwoven substrate to create a twisted compressed substrate.

Repeat use of reference characters in the present specification and drawings is intended to represent the same or analogous features or elements of the present invention.

### DETAILED DESCRIPTION

Reference now will be made to the embodiments of the invention, one or more examples of which are set forth below. Each example is provided by way of an explanation of the invention, not as a limitation of the invention. In fact, it will be apparent to those skilled in the art that various modifications and variations can be made in the invention without departing from the scope or spirit of the invention. For instance, features illustrated or described as one embodiment can be used on another embodiment to yield still a further embodiment. Thus, it is intended that the present invention cover such modifications and variations as come within the scope of the appended claims and their equivalents. It is to be understood by one of ordinary skill in the art that the present discussion is a description of exemplary embodiments only, and is not intended as limiting the broader aspects of the present invention, which broader aspects are embodied exemplary constructions.

In general, the present disclosure is directed to twisted, compressed substrates that expand upon contact with a liquid. The twisted, compressed substrates expand in the z-direction with minimal expansion in the x-direction and/or y-direction. Additionally, upon contact with a liquid, the twisted, compressed substrate unwinds from its twisted orientation to “drill” through the absorbent core of the absorbent article. Specifically, the unwinding action during expansion provides a torquing force that enables the expanding substrate to penetrate through the fluff, super-absorbent material (SAM), and/or other components of the absorbent core. Thus, the expanding, unwinding substrate can penetrate through the absorbent core instead of pushing against the absorbent core, allowing the expanding substrate to alert the wearer of an insult without significantly impairing the absorbency capacity of the absorbent core.

### I. Twisted, Compressed Substrate

The twisted, compressed substrate is constructed from a highly compressed web material. The substrate is not only compressed in the z-direction, but is also twisted either before or during compression. The present inventors have discovered that this twisted, compressed substrate will unwind from its twisted state during expansion in the z-direction upon contact with a liquid.

After compression-molding of the web material, a twisted, compressed substrate is formed that is configured to expand only in the direction of the compression forces (i.e., only in the z-direction) upon wetting. Thus, the direction of expansion upon contact with a liquid can be predisposed, allowing the direction of expansion of the twisted, compressed substrate to be predetermined when included within an absorbent article.

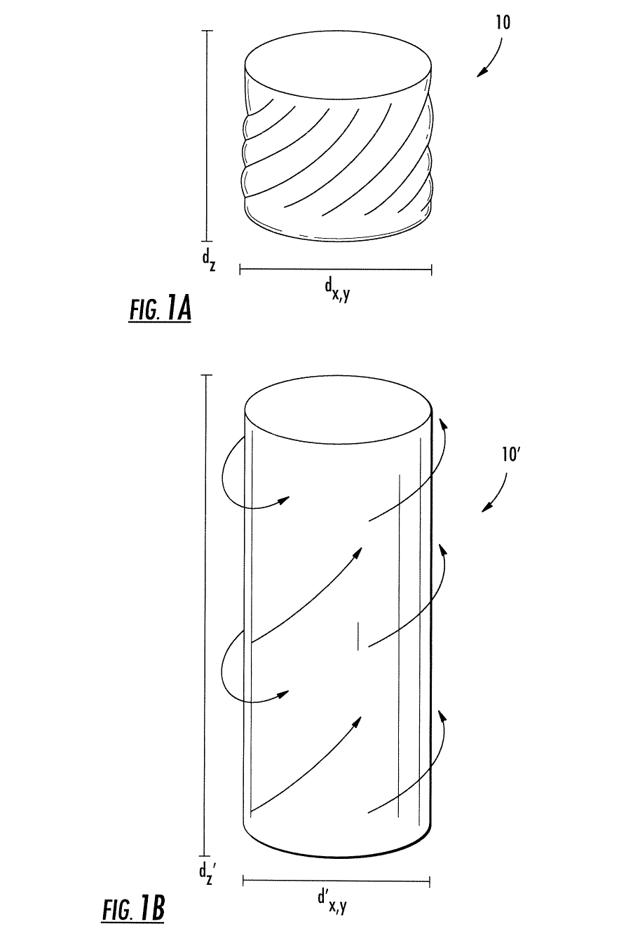

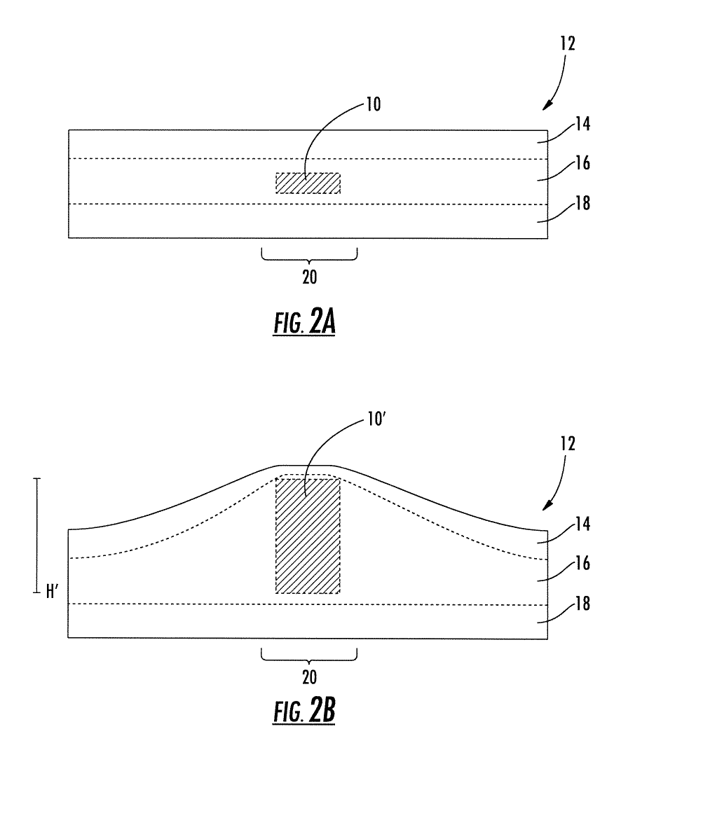

Referring to FIG. 1A, an exemplary twisted, compressed substrate 10 is shown in its dry, compressed state. The twisted, compressed substrate 10 has a compressed height d_{z} in its z-direction while still in its dry state. Upon contact with a liquid, the twisted, compressed substrate 10 expands and unwinds to be an expanded twisted, compressed substrate 10′ having an expanded height d_{z}′ (as shown in FIG. 1B). The degree of expansion in the z-direction can be predetermined by the type of material included within the twisted, compressed substrate 10 and the force asserted in forming the twisted, compressed substrate 10.

The expansion of the twisted, compressed substrate 10 is substantially 1-dimensional. Upon contact with a liquid expansion of the twisted, compressed substrate 10 occurs in the z-direction, without substantially increasing the size of the twisted, compressed substrate 10 in either the x-direction or y-direction. For example, referring to FIGS. 1A and 1B, the twisted, compressed substrate 10 is shown having a cylindrical shape, such that its size in the x- and y-directions are substantially equal (i.e., the diameter of the cylindrical twisted, compressed substrate 10). The diameter d_{x,y} of the twisted, compressed substrate 10 remains substantially unchanged after contact with a liquid causing expansion in the z-direction. Thus, the diameter d_{x,y}′ of the expanded twisted, compressed substrate 10′ shown in FIG. 1B is nearly identical to the diameter d_{x,y} of the twisted, compressed substrate 10 shown in FIG. 1A (e.g., d_{x,y}′≦1.1 times d_{x,y}).

The expansion of the twisted, compressed substrate can be stated as an “expansion ratio” comparing of the degree of expansion in the z-direction compared to the degree of expansion in both the x- and y-directions (i.e., d_{z}′ divided by d_{z} compared to d_{x,y}′ divided by d_{x,y}). In particular embodiments, the twisted, compressed substrate can expand more than about 2:1.1 in the z-direction compared to the x- and y-directions, such as greater than 3:1.1, and from about 5:1.1 to about 10:1.1. For example, the expansion ration can be greater than about 2:1.05, such as greater than about 3:1.05, such as from about 5:1.05 to about 10:1.05.

For example, the twisted, compressed substrate 10 suitably expands to at least about 2 times its original height d_{z} in the z-direction when dry (i.e., expands 200%), and more suitably it expands to at least about 3 times the original height d_{z} when dry (i.e., expands 300%). For example, in some embodiments, the expanded twisted, compressed substrate 10′ can have a thickness or height d_{z}′ that is from about 5 times to about 10 times its original height d_{z} (i.e., expands from about 500% to about 1000%).

In one particular embodiment, the diameter d_{x,y}′ of the expanded twisted, compressed substrate 10′ can be less than about 110% of the diameter d_{x,y} of the twisted, compressed substrate 10 in a dry state (i.e., less than about 1.1 times the original diameter d_{x,y}), such as from 100% (i.e., unchanged in diameter upon contact with a liquid in the x- and y-directions) to about 107% (i.e., about 1.07 times the original diameter d_{x,y}). For instance, the diameter d_{x,y}′ of the expanded twisted, compressed substrate 10′ can be from about 100.5% to about 105% of the diameter d_{x,y} of the twisted, compressed substrate 10 in a dry state.

Of course, the twisted, compressed substrate 10 can be molded into any other shape, including but not limited to cuboids, cubes, cones, etc. No matter the particular shape of the twisted, compressed substrate 10, the dimensions in the x- and y-directions do not substantially increase upon contact with a liquid. Suitable twisted, compressed substrates are disclosed in U.S. patent application Ser. Nos. 11/955,916 and 11/955,937 filed on Dec. 13, 2007, the disclosures of which are incorporated in their entirety herein.

The twisted, compressed substrate 10 is configured to expand to the expanded twisted, compressed substrate 10′ nearly immediately upon contact with a small amount of a liquid. For example, the 1-dimensional expansion can occur within about 10 seconds of the twisted, compressed substrate 10 contacting a liquid, such expanding in less than about 5 seconds. In some embodiments, the 1-dimensional expansion of the twisted, compressed substrate 10 can occur from about 1 second to about 5 seconds, such as from about 1 second to about 3 seconds. Thus, the wearer of the absorbent article can be immediately alerted upon the first insult of the absorbent article.

In order to initiate the expansion of the twisted, compressed substrate 10, the twisted, compressed substrate 10 is configured to expand upon contact with a small amount of liquid. This amount of liquid need not completely saturate the twisted, compressed substrate 10. Of course, the amount of liquid necessary to cause complete expansion of the twisted, compressed substrate 10 to the expanded twisted, compressed substrate 10′ can vary with the size of the twisted, compressed substrate 10. However, when used in an absorbent article, the twisted, compressed substrate 10 is configured, in most embodiments, to expand upon contact with greater than about 0.1 milliliters (mL), such as from about 0.5 mL to about 7 mL, and from about 1 mL to about 5 mL. At these liquid levels, the twisted, compressed substrate 10 can at least double in height in the z-direction with an expansion ratio of at least 2:1.1, as stated above.

The twisted, compressed substrate offer the moisture triggered z-directional expansion with a significant amount of energy. Specifically, the twisted, compressed substrate can expand in the z-direction with an exerted force up to about 16 pounds per square inch (psi), such from about 10 psi to about 15 psi. Thus, the twisted, compressed substrate can press against the skin of the wearer with sufficient force to alert the wearer that an insult has occurred. Additionally, by unwinding from its twisted state during expansion upon contact with a liquid, the expanding substrate can drill through the absorbent core of the absorbent article to avoid asserting undue pressure on the core.

The web material that is compressed to form the twisted, compressed substrate can be a nonwoven web of fibers. Although the particular type of fiber is not a limitation of the invention, some fibers are particularly suitable for forming the twisted, compressed substrate 10 to be included within an absorbent article. The fibers may be, for example, any combination of synthetic or pulp fibers. The selected average fiber length and denier will generally depend on a variety of factors and desired processing steps.

In one embodiment, a substantial portion of the fibers may be cellulosic pulp staple fibers. Pulp fibers may be utilized to reduce costs, as well as impart other benefits to the twisted, compressed substrate 10, such as improved absorbency. Some examples of suitable cellulosic fiber sources include virgin wood fibers, such as thermomechanical, bleached and unbleached pulp fibers. Pulp fibers may have a high-average fiber length, a low-average fiber length, or mixtures of the same. Some examples of suitable high-average length pulp fibers include northern softwood, southern softwood, redwood, red cedar, hemlock, pine (e.g., southern pines), spruce (e.g., black spruce), combinations thereof, and so forth. Some examples of suitable low-average fiber length pulp fibers may include certain virgin hardwood pulps and secondary (i.e. recycled) fiber pulp from sources such as, for example, newsprint, reclaimed paperboard, and office waste. Hardwood fibers, such as eucalyptus, maple, birch, aspen, and so forth, may also be used as low-average length pulp fibers. These pulp fibers can be formed into a nonwoven web (e.g., a tissue web) according to any process (e.g., wetlaid, airlaid, bonded carded process, etc.).

In one particular embodiment, the web is a non-woven web of rayon material. In particular, the rayon material can be manufactured by a spun lace method in which a web is formed out of viscose rayon and fibers are coupled using a high-pressure water stream.

Alternatively, a majority of the fibers of the nonwoven web may be formed from synthetic polymers. Synthetic fibers can be formed into nonwoven fabrics or webs from many processes such as for example, meltblowing processes, spunbonding processes, bonded carded web processes, etc.

“Meltblown fibers” refers to fibers formed by extruding a molten thermoplastic material through a plurality of fine, usually circular, die capillaries as molten fibers into converging high velocity gas (e.g. air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin et al., which is incorporated herein in its entirety by reference thereto for all purposes. Generally speaking, meltblown fibers may be microfibers that may be continuous or discontinuous, are generally smaller than 10 microns in diameter, and are generally tacky when deposited onto a collecting surface.

“Spunbonded fibers” refers to small diameter substantially continuous fibers that are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spun-bonded nonwoven webs is described and illustrated, for example, in U.S. Pat. Nos. 4,340,563 to Appel, et al., 3,692,618 to Dorschner, et al., 3,802,817 to Matsuki, et al., 3,338,992 to Kinney, 3,341,394 to Kinney, 3,502,763 to Hartman, 3,502,538 to Petersen, 3,542,615 to Dobo, et al., and 5,382,400 to Pike, et al., which are incorporated herein in their entirety by reference thereto for all purposes. Spunbond fibers are generally not tacky when they are deposited onto a collecting surface. Spunbond fibers can sometimes have diameters less than about 40 microns, and are often between about 5 to about 20 microns.

Exemplary synthetic polymers for use in forming nonwoven web may include, for instance, polyolefins, e.g., polyethylene, polypropylene, polybutylene, etc.; polytetrafluoroethylene; polyesters, e.g., polyethylene terephthalate and so forth; polyvinyl acetate; polyvinyl chloride acetate; polyvinyl butyral; acrylic resins, e.g., polyacrylate, polymethylacrylate, polymethylmethacrylate, and so forth; polyamides, e.g., nylon; polyvinyl chloride; polyvinylidene chloride; polystyrene; polyvinyl alcohol; polyurethanes; polylactic acid; copolymers thereof; and so forth. If desired such as those described above, may also be employed. It should be noted that the polymer(s) may also contain other additives, such as processing aids or treatment compositions to impart desired properties to the fibers, residual amounts of solvents, pigments or colorants, and so forth.

Monocomponent and/or multicomponent fibers may be used to form the nonwoven web. Monocomponent fibers are generally formed from a polymer or blend of polymers extruded from a single extruder. Multicomponent fibers are generally formed from two or more polymers (e.g., bicomponent fibers) extruded from separate extruders. The polymers may be arranged in substantially constantly positioned distinct zones across the cross-section of the fibers. The components may be arranged in any desired configuration, such as sheath-core, side-by-side, pie, island-in-the-sea, three island, bull's eye, or various other arrangements known in the art. Various methods for forming multicomponent fibers are described in U.S. Pat. Nos. 4,789,592 to Taniguchi et al. and 5,336,552 to Strack, et al., 5,108,820 to Kaneko, et al., 4,795,668 to Kruege, et al., 5,382,400 to Pike, et al., 5,336,552 to Strack, et al., and 6,200,669 to Marmon, et al., which are incorporated herein in their entirety by reference thereto for all purposes. Multicomponent fibers having various irregular shapes may also be formed, such as described in U.S. Pat. Nos. 5,277,976 to Hogle, et al., 5,162,074 to Hills, 5,466,410 to Hills, 5,069,970 to Largman, et al., and 5,057,368 to Largman, et al., which are incorporated herein in their entirety by reference thereto for all purposes.

Although any combination of polymers may be used, the polymers of the multicomponent fibers are typically made from thermoplastic materials with different glass transition or melting temperatures where a first component (e.g., sheath) melts at a temperature lower than a second component (e.g., core). Softening or melting of the first polymer component of the multicomponent fiber allows the multicomponent fibers to form a tacky skeletal structure, which upon cooling, stabilizes the fibrous structure. For example, the multicomponent fibers may have from about 5% to about 80%, and in some embodiments, from about 10% to about 60% by weight of the low melting polymer. Further, the multicomponent fibers may have from about 95% to about 20%, and in some embodiments, from about 90% to about 40%, by weight of the high melting polymer. Some examples of known sheath-core bicomponent fibers available from KoSa Inc. of Charlotte, N.C. under the designations T-255 and T-256, both of which use a polyolefin sheath, or T-254, which has a low melt co-polyester sheath. Still other known bicomponent fibers that may be used include those available from the Chisso Corporation of Moriyama, Japan or Fibervisions LLC of Wilmington, Del.

Fibers of any desired length may be employed, such as staple fibers, continuous fibers, etc. In one particular embodiment, for example, staple fibers may be used that have a fiber length in the range of from about 1 to about 150 millimeters, in some embodiments from about 5 to about 50 millimeters, in some embodiments from about 10 to about 40 millimeters, and in some embodiments, from about 10 to about 25 millimeters. Although not required, carding techniques may be employed to form fibrous layers with staple fibers as is well known in the art. For example, fibers may be formed into a carded web by placing bales of the fibers into a picker that separates the fibers. Next, the fibers are sent through a combing or carding unit that further breaks apart and aligns the fibers in the machine direction so as to form a machine direction-oriented fibrous nonwoven web. The carded web may then be bonded using known techniques to form a bonded carded nonwoven web.

If desired, the nonwoven web may have a multi-layer structure. The other layers can be other nonwoven webs, films, and the like. For example, in one embodiment, at least two nonwoven webs can be combined to form a nonwoven laminate. Suitable multi-layered materials may include, for instance, spunbond/meltblown/spunbond (SMS) laminates and spunbond/meltblown (SM) laminates. Various examples of suitable SMS laminates are described in U.S. Pat. Nos. 4,041,203 to Brock et al.; 5,213,881 to Timmons, et al.; 5,464,688 to Timmons, et al.; 4,374,888 to Bornslaeger; 5,169,706 to Collier, et al.; and 4,766,029 to Brock et al., which are incorporated herein in their entirety by reference thereto for all purposes. In addition, commercially available SMS laminates may be obtained from Kimberly-Clark Corporation under the designations Spunguard® and Evolution®.

Another example of a multi-layered structure is a spunbond web produced on a multiple spin bank machine in which a spin bank deposits fibers over a layer of fibers deposited from a previous spin bank. Such an individual spunbond nonwoven web may also be thought of as a multi-layered structure. In this situation, the various layers of deposited fibers in the nonwoven web may be the same, or they may be different in basis weight and/or in terms of the composition, type, size, level of crimp, and/or shape of the fibers produced. As another example, a single nonwoven web may be provided as two or more individually produced layers of a spunbond web, a carded web, etc., which have been bonded together to form the nonwoven web. These individually produced layers may differ in terms of production method, basis weight, composition, and fibers as discussed above.

A nonwoven web constructed from synthetic fibers may also contain an additional fibrous component such that it is considered a composite. For example, a nonwoven web may be entangled with another fibrous component using any of a variety of entanglement techniques known in the art (e.g., hydraulic, air, mechanical, etc.). In one embodiment, the nonwoven web is integrally entangled with cellulosic fibers using hydraulic entanglement. Hydraulically entangled nonwoven webs of staple length and continuous fibers are disclosed, for example, in U.S. Pat. Nos. 3,494,821 to Evans and 4,144,370 to Boulton, which are incorporated herein in their entirety by reference thereto for all purposes. Hydraulically entangled composite nonwoven webs of a continuous fiber nonwoven web and a pulp layer are disclosed, for example, in U.S. Pat. Nos. 5,284,703 to Everhart, et al. and 6,315,864 to Anderson, et al., which are incorporated herein in their entirety by reference thereto for all purposes.

No matter the particular construction of the nonwoven web, the web is compression molded into a twisted, compressed substrate 10 configured to expand 1-dimensionally. The 1-dimensional expansion generally occurs in the direction of the compression forces exerted during the formation of the twisted, compressed substrate 10. Thus, one of ordinary skill in the art would be able to form a twisted, compressed substrate 10 having any desired shape and any desired expansion parameters. Additionally, the web is twisted either prior to or during compression.

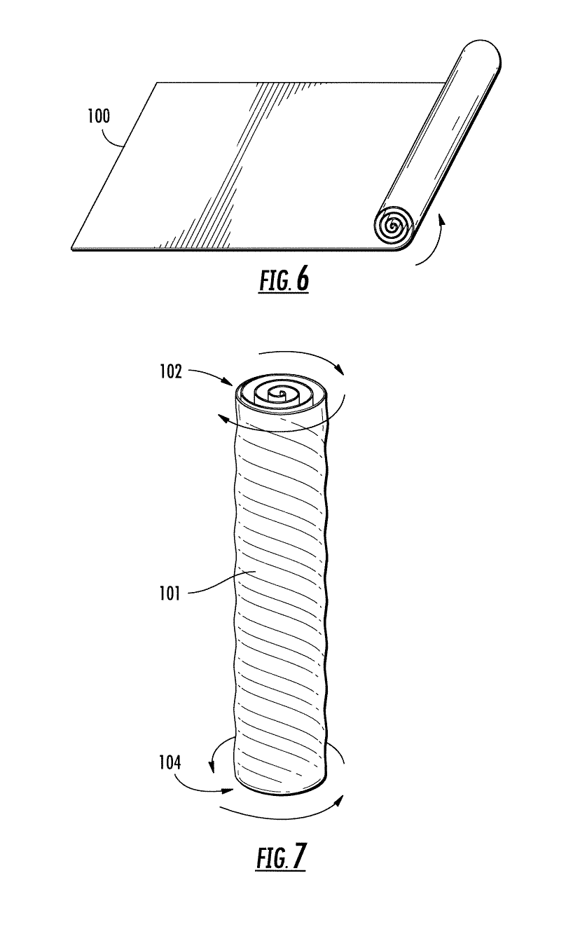

In one embodiment, the compressed web materials can be formed by first folding or rolling the web material into a tube-like shape, such that the web material is generally longer in the z-direction than in the x- and y-directions. For example, FIG. 6 shows a web material 100 being rolled into a cylindrical tube. This folded or rolled web material can then be twisted along its length. Referring to FIG. 7, the top section 102 of the rolled cylindrical tube 101 of web material 100 is twisted in a direction opposite to the bottom section 104. The amount of twist in the rolled cylindrical tube 101 can be measured as the relative amount of twist between the top section 102 and the bottom section 104. The amount of twisting can vary depending on the amount of unwinding desired. In one embodiment, the web material can be twisted at least about 360° (i.e., the top section 102 is rotated at least once relative to the bottom section), such as at least about 540°. In some embodiments, the web material can be twisted at least about 720°, such as from about 1080° to about 1800°.

In one particular embodiment, the web can be wetted (e.g., saturated with a liquid, such as water), twisted the desired amount, and then dried. Drying the web is in a twisted state holds the web in its twisted state, such that the twisted, rolled web can be placed into a compression barrel without significantly unwinding before compression can begin.

The twisted material can then be placed into an elongated barrel such that the longer z-direction of the twisted web is parallel with the length of the barrel. The shape of the barrel in the x- and y-directions corresponds to the shape of the resulting twisted, compressed substrate 10. For example, to make the twisted, compressed substrate 10 shown in FIG. 1A, the barrel shape is cyclical such that the x- and y-directions of the barrel define a circle (or oval). Alternatively, the barrel shape can define any desired shape in the x- and y-directions to produce the twisted, compressed substrate 10 in the desired shape.

After placement in the barrel, the folded or rolled web is subjected to a compression force in a direction of the elongation of the barrel (i.e., the z-direction). This compression force is sufficient to compress the folded or rolled web into a twisted, compressed substrate 10 that will retain its initial shape until exposure to a liquid. That is, the disposable tissue should be subjected to compression molding under a pressure within a predetermined pressure range that varies according to the shape, configuration, and chemical construction of the web as described above. However, if the web is pressed under a pressure within the predetermined pressure range, it is compressed at a compressibility (ΔV/V) in a range of 0.4 to 0.6. Here, the compressibility (ΔV/V) represents a ratio of the amount of volume change (ΔV) in the twisted, compressed substrate 10 to the volume (V) of the uncompressed web. The amount of volume change means the difference between the volume (V) of the uncompressed web and the volume of the twisted, compressed substrate 10.

For example, when making a twisted, compressed substrate 10 shaped as in FIG. 1A with a diameter d_{x,y} of about 2 cm and a height d_{z} of about 1 cm from a web. The web can have any initial size, such as less than about 20 cm×20 cm, such as from about 5 cm×5 cm to about 15 cm×15 cm. In one particular embodiment, the web can have an initial size of about 10×10 cm. The compression force can be apply a pressure to the folded or rolled tissue web of about 95 kiloNewton (kN) to about 300 kN, such as from about 145 kN to about 250 kN. In one particular embodiment the compression force can be from about 190 kN to about 200 kN in the z-direction.

Although the apparatus for forming the twisted, compressed substrate 10 can vary, a particularly suitable apparatus can include a cylindrical molding barrel having a longitudinal, through passage. The molding barrel can be supported on a table such that both end portions of the through passage of the molding barrel are exposed to the outside. An upper press can be installed vertically movably above the table and having a pressing rod to be inserted into the through passage of the molding barrel when the upper press moves downwardly. A lower press can also be installed vertically movably below the table and having a supporting rod to be inserted into the through passage of the molding barrel when the lower press moves upwardly.

In this set up, the upper press can include a power source for pressing the folded or rolled web received in the through passage. The supporting rod of the lower press closes an entrance of the through passage of the molding barrel to compression-mold the folded or rolled web and opens the entrance of the through passage to discharge the twisted, compressed substrate 10 from the through passage. The twisted, compressed substrate 10 is molded to have a shape that is the same as a space defined by the through passage of the molding barrel, the supporting rod of the lower press, and the pressing rod of the upper press. In a state where the entrance of the through passage of the molding barrel is opened, the twisted, compressed substrate 10 is discharged from the through passage by the upper press moving downwardly.

In one particular embodiment, the compressed web materials can be made with the compression molding apparatus and methods described in International Publication No. WO 200/082448 A1 of Lee, et al., the disclosure of which is incorporated herein by reference.

Alternatively, the compression force can add a twisting component. For example, when using a cylindrical barrel, the pressing rod and the elongated barrel can be threaded such that that pressing rod twists while compressing the web material. In this embodiment, the pressing rod can have a web material contacting surface that engages the web material and twists it during compression.

### II. Absorbent Articles

In one embodiment, the twisted, compressed substrate can be included within an absorbent article as a wetness indicator, such as disclosed in U.S. patent application Ser. Nos. 11/955,916 and 11/955,937 filed on Dec. 13, 2007, the disclosures of which are incorporated in their entirety herein. Upon wetting, the twisted, compressed substrate expands in the z-direction, without any significant expansion in the x- or y-directions, and presses against the skin of the wearer. Also, the twisted, compressed substrate unwinds upon wetting. This unwinding provides an additional torquing action to the z-direction expansion enabling the expanding substrate to penetrate through the absorbent core of the article avoiding putting too much pressure on the components of the absorbent core. Additionally, since there is minimal x- and y-direction expansion, the twisted, compressed substrate of the present invention does not substantially alter or interfere with the absorbent capabilities of the absorbent article by pressing against the absorbent core in the x- and y-directions. Thus, the twisted, compressed substrate can be included within conventional absorbent articles without significantly sacrificing the absorbency characteristics of the article.

The twisted, compressed substrate of the present invention is configured to expand toward the skin of the wearer (i.e., in the z-direction of the absorbent article perpendicular to the plane of the absorbent article) upon contact with a liquid. That is, the twisted, compressed substrate does not substantially expand in any direction parallel with the plane of the article (i.e., the x- and y-directions). As such, the twisted, compressed substrate does not significantly interfere with the absorbent capabilities of the absorbent article.

The twisted, compressed substrate 10 can be included in an absorbent article as a tactile cue to indicate to the wearer that an insult has occurred. Upon wetting, the twisted, compressed substrate 10 can expand to press against the wearers skin. The term “absorbent article” generally refers to any article capable of absorbing water or other fluids. Examples of some absorbent articles include, but are not limited to, personal care absorbent articles, such as diapers, training pants, absorbent underpants, incontinence articles, feminine hygiene products (e.g., sanitary napkins), swim wear, baby wipes, and so forth; medical absorbent articles, such as garments, fenestration materials, underpads, bedpads, bandages, absorbent drapes, and medical wipes; food service wipers; clothing articles; and so forth. Materials and processes suitable for forming such absorbent articles are well known to those skilled in the art. Typically, absorbent articles include a substantially liquid-impermeable layer (e.g., outer cover), a liquid-permeable layer (e.g., bodyside liner, surge layer, etc.), and an absorbent core.

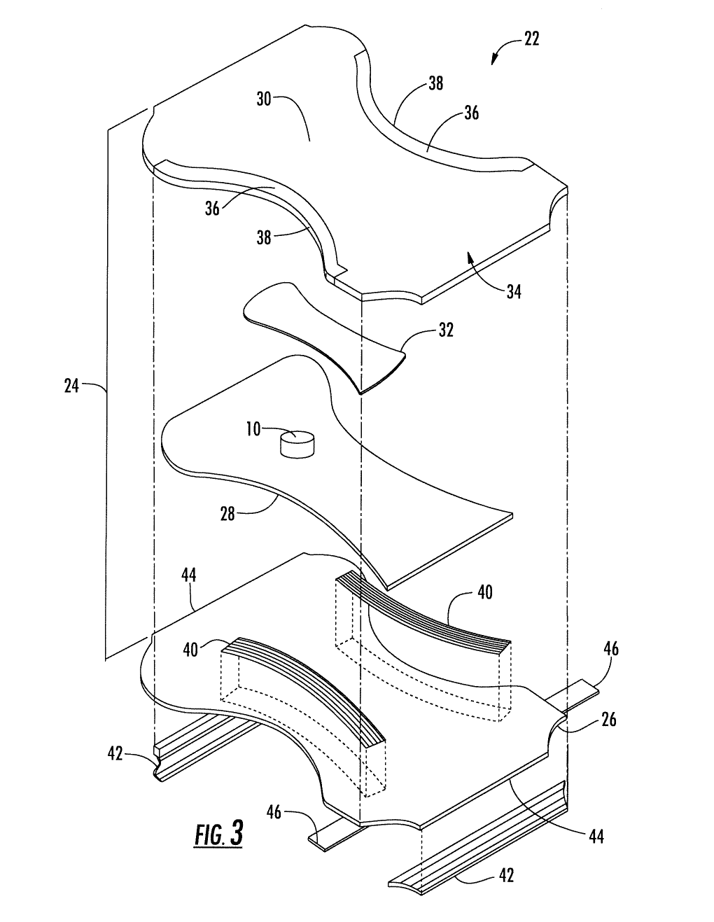

With particular reference now to FIG. 2A, a twisted, compressed substrate 10 is suitably disposed between the liquid-permeable layer 102 and the liquid-impermeable layer 106 so that the twisted, compressed substrate 10 is substantially imperceptible to the wearer prior to the first insult of the absorbent article 100 by liquid body exudates (e.g., urine, menses, feces).

The twisted, compressed substrate 10 can be positioned in the crotch region of the absorbent article (e.g., within the middle third of the absorbent article in both the longitudinal and lateral directions). However, it is contemplated that the longitudinal position of the twisted, compressed substrate 10 within the crotch region (e.g., the middle third of the length of the absorbent article) may be dependant on the type of absorbent article and/or the gender of the intended wearer.

While a single twisted, compressed substrate 10 is shown in the illustrated embodiment of FIG. 2A, it is contemplated that additional twisted, compressed substrates 10 may be used to further enhance the signal to the wearer. For example, additional twisted, compressed substrates 10 may be necessary for larger absorbent articles for whom the resistive force provided by a single twisted, compressed substrate 10 may be insufficient to alert the wearer to insult of the absorbent article 100.

The thickness, or height H, of the twisted, compressed substrate 10 when dry is suitably in the range of about 2 mm to about 20 mm, and more suitably in the range of about 5 mm to about 15 mm, such as about 10 mm. Upon absorption of a liquid, the thickness, or height H′, of the expanded twisted, compressed substrate 10′ suitably expands to at least about 2 times its original height H when dry, and more suitably it expands to at least about 3 times the height H when dry. For example, in some embodiments, the expanded twisted, compressed substrate 10′ can have a thickness or height H′ that is from about 5 times to about 10 times its original height H. This 1-dimensional expansion is generally achieved according to the expansion ratio described above, with contact of greater than 0.1 mL of a liquid.

At the relatively small initial height H, the twisted, compressed substrate 10 does not substantially interfere with the flexibility of the absorbent article, nor does the twisted, compressed substrate 10 substantially interfere with the absorbent capacity of the absorbent core 16. For example, the twisted, compressed substrate 10 can have a width of less than about 33% of the width of the absorbent core, such as less than about 25%. In most embodiments, the twisted, compressed substrate 10 has a width and length in the x- and y-directions of less than about 5 centimeters (cm), such as from about 1 cm to about 4 cm, and from about 2 cm to about 3 cm.

Various embodiments of an absorbent article that may be formed according to the present invention, such as diapers, incontinence articles, sanitary napkins, diaper pants, feminine napkins, children's training pants, and so forth. Various configurations of a diaper are described in U.S. Pat. Nos. 4,798,603 to Meyer et al.; 5,176,668 to Bemardin; 5,176,672 to Bruemmer et al.; 5,192,606 to Proxmire et al.; and 5,509,915 to Hanson et al., as well as U.S. Patent Application Pub. No. 2003/120253 to Wentzel, et al., all of which are incorporated herein in their entirety by reference thereto for all purposes.

Various embodiments of an absorbent article that may be formed according to the present invention will now be described in more detail. However, as noted above, the invention may be embodied in any type of absorbent articles, such as diapers, incontinence articles, sanitary napkins, diaper pants, feminine napkins, children's training pants, and so forth.

A. Diapers, Training Pants, and Incontinent Articles

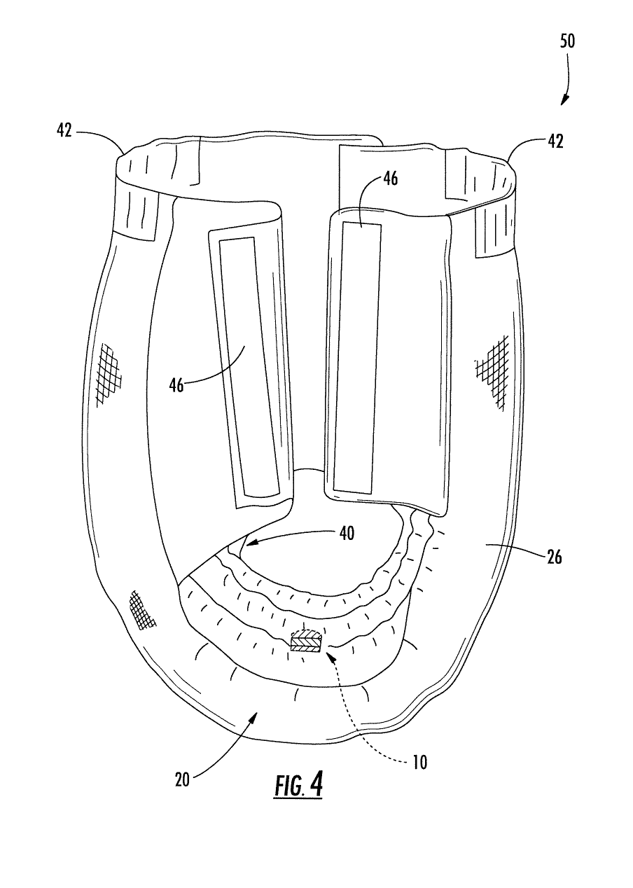

For purposes of illustration only, an absorbent article is shown in FIG. 3 as a diaper 22. In the illustrated embodiment, the diaper 22 is shown as having an hourglass shape in an unfastened configuration. However, other shapes may of course be utilized, such as a generally rectangular shape, T-shape, or I-shape. As shown, the diaper 22 includes a chassis 24 formed by various components, including an outer cover 26, bodyside liner 30, absorbent core 28, and surge layer 32. It should be understood, however, that other layers may also be used in the present invention. Likewise, one or more of the layers referred to in FIG. 3 may also be eliminated in certain embodiments of the present invention.

The outer cover 26 is typically formed from a material that is substantially impermeable to liquids. For example, the outer cover 26 may be formed from a thin plastic film or other flexible liquid-impermeable material. In one embodiment, the outer cover 26 is formed from a polyethylene film having a thickness of from about 0.01 millimeter to about 0.05 millimeter. If a more cloth-like feeling is desired, the outer cover 26 may be formed from a polyolefin film laminated to a nonwoven web. For example, a stretch-thinned polypropylene film having a thickness of about 0.015 millimeter may be thermally laminated to a spunbond web of polypropylene fibers. The polypropylene fibers may have a denier per filament of about 1.5 to 2.5, and the nonwoven web may have a basis weight of about 17 grams per square meter. The outer cover 26 may also include bicomponent fibers, such as polyethylene/polypropylene bicomponent fibers. In addition, the outer cover 26 may also contain a material that is impermeable to liquids, but permeable to gases and water vapor (i.e., “breathable”). This permits vapors to escape from the absorbent core 28, but still prevents liquid exudates from passing through the outer cover 26.

The diaper 22 also includes a bodyside liner 30. The bodyside liner 30 is generally employed to help isolate the wearer's skin from liquids held in the absorbent core 28. For example, the liner 30 presents a bodyfacing surface that is typically compliant, soft feeling, and non-irritating to the wearer's skin. Typically, the liner 30 is also less hydrophilic than the absorbent core 28 so that its surface remains relatively dry to the wearer. The liner 30 may be liquid-permeable to permit liquid to readily penetrate through its thickness. The bodyside liner 30 may be formed from a wide variety of materials, such as porous foams, reticulated foams, apertured plastic films, natural fibers (e.g., wood or cotton fibers), synthetic fibers (e.g., polyester or polypropylene fibers), or a combination thereof. In some embodiments, woven and/or nonwoven fabrics are used for the liner 30. For example, the bodyside liner 30 may be formed from a meltblown or spunbonded web of polyolefin fibers. The liner 30 may also be a bonded-carded web of natural and/or synthetic fibers. The liner 30 may further be composed of a substantially hydrophobic material that is optionally treated with a surfactant or otherwise processed to impart a desired level of wettability and hydrophilicity. The surfactant may be applied by any conventional method, such as spraying, printing, brush coating, foaming, and so forth. When utilized, the surfactant may be applied to the entire liner 30 or may be selectively applied to particular sections of the liner 30, such as to the medial section along the longitudinal centerline of the diaper. The liner 30 may further include a composition that is configured to transfer to the wearer's skin for improving skin health. Suitable compositions for use on the liner 30 are described in U.S. Pat. No. 6,149,934 to Krzysik et al., which is incorporated herein in its entirety by reference thereto for all purposes.

As illustrated in FIG. 3, the diaper 22 may also include a surge layer 32 that helps to decelerate and diffuse surges or gushes of liquid that may be rapidly introduced into the absorbent core 28. Desirably, the surge layer 32 rapidly accepts and temporarily holds the liquid prior to releasing it into the storage or retention portions of the absorbent core 28. In the illustrated embodiment, for example, the surge layer 32 is interposed between an inwardly facing surface of the bodyside liner 30 and the absorbent core 28. Alternatively, the surge layer 32 may be located on an outwardly facing surface 34 of the bodyside liner 30. The surge layer 32 is typically constructed from highly liquid-permeable materials. Suitable materials may include porous woven materials, porous nonwoven materials, and apertured films. Some examples include, without limitation, flexible porous sheets of polyolefin fibers, such as polypropylene, polyethylene or polyester fibers; webs of spunbonded polypropylene, polyethylene or polyester fibers; webs of rayon fibers; bonded carded webs of synthetic or natural fibers or combinations thereof. Other examples of suitable surge layers 32 are described in U.S. Pat. No. 5,486,166 to Ellis, et al. and U.S. Pat. No. 5,490,846 to Ellis, et al., which are incorporated herein in their entirety by reference thereto for all purposes.

Besides the above-mentioned components, the diaper 22 may also contain various other components as is known in the art. For example, the diaper 22 may also contain a substantially hydrophilic tissue wrapsheet (not illustrated) that helps maintain the integrity of the fibrous structure of the absorbent core 28. The tissue wrapsheet is typically placed about the absorbent core 28 over at least the two major facing surfaces thereof, and composed of an absorbent cellulosic material, such as creped wadding or a high wet-strength tissue. The tissue wrapsheet may be configured to provide a wicking layer that helps to rapidly distribute liquid over the mass of absorbent fibers of the absorbent core 28. The wrapsheet material on one side of the absorbent fibrous mass may be bonded to the wrapsheet located on the opposite side of the fibrous mass to effectively entrap the absorbent core 28.

Furthermore, the diaper 22 may also include a ventilation layer (not shown) that is positioned between the absorbent core 28 and the outer cover 26. When utilized, the ventilation layer may help insulate the outer cover 26 from the absorbent core 28, thereby reducing dampness in the outer cover 26. Examples of such ventilation layers may include breathable laminates (e.g., nonwoven web laminated to a breathable film), such as described in U.S. Pat. No. 6,663,611 to Blaney, et al., which is incorporated herein in its entirety by reference thereto for all purpose.

As representatively illustrated in FIG. 3, the diaper 22 may also include a pair of containment flaps 36 that are configured to provide a barrier and to contain the lateral flow of body exudates. The containment flaps 36 may be located along the laterally opposed side edges 38 of the bodyside liner 30 adjacent the side edges of the absorbent core 28. The containment flaps 36 may extend longitudinally along the entire length of the absorbent core 28, or may only extend partially along the length of the absorbent core 28. When the containment flaps 36 are shorter in length than the absorbent core 28, they may be selectively positioned anywhere along the side edges 38 of diaper 22 in a crotch region 10. In one embodiment, the containment flaps 36 extend along the entire length of the absorbent core 28 to better contain the body exudates. Such containment flaps 36 are generally well known to those skilled in the art. For example, suitable constructions and arrangements for the containment flaps 36 are described in U.S. Pat. No. 4,704,116 to Enloe, which is incorporated herein in its entirety by reference thereto for all purposes.

The diaper 22 may include various elastic or stretchable materials, such as a pair of leg elastic members 40 affixed to the side edges 38 to further prevent leakage of body exudates and to support the absorbent core 28. In addition, a pair of waist elastic members 42 may be affixed to longitudinally opposed waist edges 44 of the diaper 22. The leg elastic members 40 and the waist elastic members 42 are generally adapted to closely fit about the legs and waist of the wearer in use to maintain a positive, contacting relationship with the wearer and to effectively reduce or eliminate the leakage of body exudates from the diaper 22. As used herein, the terms “elastic” and “stretchable” include any material that may be stretched and return to its original shape when relaxed. Suitable polymers for forming such materials include, but are not limited to, block copolymers of polystyrene, polyisoprene and polybutadiene; copolymers of ethylene, natural rubbers and urethanes; etc. Particularly suitable are styrene-butadiene block copolymers sold by Kraton Polymers of Houston, Tex. under the trade name Kraton®. Other suitable polymers include copolymers of ethylene, including without limitation ethylene vinyl acetate, ethylene methyl acrylate, ethylene ethyl acrylate, ethylene acrylic acid, stretchable ethylene-propylene copolymers, and combinations thereof. Also suitable are coextruded composites of the foregoing, and elastomeric staple integrated composites where staple fibers of polypropylene, polyester, cotton and other materials are integrated into an elastomeric meltblown web. Certain elastomeric single-site or metallocene-catalyzed olefin polymers and copolymers are also suitable for the side panels.

The diaper 22 may also include one or more fasteners 46. For example, two flexible fasteners 46 are illustrated in FIG. 3 on opposite side edges of waist regions to create a waist opening and a pair of leg openings about the wearer. The shape of the fasteners 46 may generally vary, but may include, for instance, generally rectangular shapes, square shapes, circular shapes, triangular shapes, oval shapes, linear shapes, and so forth. The fasteners may include, for instance, a hook material. In one particular embodiment, each fastener 46 includes a separate piece of hook material affixed to the inside surface of a flexible backing.

The various regions and/or components of the diaper 22 may be assembled together using any known attachment mechanism, such as adhesive, ultrasonic, thermal bonds, etc. Suitable adhesives may include, for instance, hot melt adhesives, pressure-sensitive adhesives, and so forth. When utilized, the adhesive may be applied as a uniform layer, a patterned layer, a sprayed pattern, or any of separate lines, swirls or dots. In the illustrated embodiment, for example, the outer cover 26 and bodyside liner 30 are assembled to each other and to the absorbent core 28 using an adhesive. Alternatively, the absorbent core 28 may be connected to the outer cover 26 using conventional fasteners, such as buttons, hook and loop type fasteners, adhesive tape fasteners, and so forth. Similarly, other diaper components, such as the leg elastic members 40, waist elastic members 42 and fasteners 46, may also be assembled into the diaper 22 using any attachment mechanism.

Although various configurations of a diaper have been described above, it should be understood that other diaper and absorbent article configurations are also included within the scope of the present invention. For instance, other suitable diaper configurations are described in U.S. Pat. Nos. 4,798,603 to Meyer et al.; 5,176,668 to Bemardin; 5,176,672 to Bruemmer et al.; 5,192,606 to Proxmire et al.; and 5,509,915 to Hanson et al., as well as U.S. Patent Application Pub. No. 2003/120253 to Wentzel, et al., all of which are incorporated herein in their entirety by reference thereto for all purposes.

According to the present invention, the twisted, compressed substrate 10 can be positioned between the outer cover 26 and the bodyside liner 30 in the diaper 22. In one particular embodiment, the twisted, compressed substrate 10 can be located between the absorbent core 28 and the bodyside liner 30. For example, in the diaper 22 shown in FIG. 3, the twisted, compressed substrate 10 is positioned between the absorbent core 28 and the surge layer 32 in the target zone (e.g., the crotch region) of the diaper 22. This particular orientation allows the twisted, compressed substrate 10 to contact a sufficient amount of liquid upon insult of the diaper 22, while still dissipating the liquid to the absorbent core 28 for absorption. However, the twisted, compressed substrate 10 could be located on either side of the surge layer 32.

Additionally, the lateral placement of the twisted, compressed substrate 10 can vary according to the gender of the intended wearer. For example, placement of the twisted, compressed substrate 10 in a more forward location within the crotch region 20 may be appropriate for boys, while placement in a more central location within the crotch region 20 may be more appropriate for girls. It is also understood that the twisted, compressed substrate 10 may be positioned other than in the crotch region 26 without departing from the scope of the present invention, as long as the wetness indicator is suitably positioned so as to become wet and perceptible by a wearer upon insult of the pants by liquid body exudates.

Alternatively, a pair of twisted, compressed substrates 10 may also be used in a configuration wherein one wetness indicator is positioned longitudinally where it is more likely to become wet upon urination by boys and the other wetness indicator is positioned longitudinally where it is more likely to become wet upon urination by girls, thereby accounting for differences between the target wetting areas of boys and girls.

In another embodiment, a training pant 50 can be constructed with a twisted, compressed substrate 10 within the crotch region 20, as shown in FIG. 4. The training pant 50 can have a similar construction than the diaper 22 described above. As stated, the twisted, compressed substrate 10 of the illustrated embodiment is small enough to not take up a substantial part of the crotch region.

B. Absorbent Pads

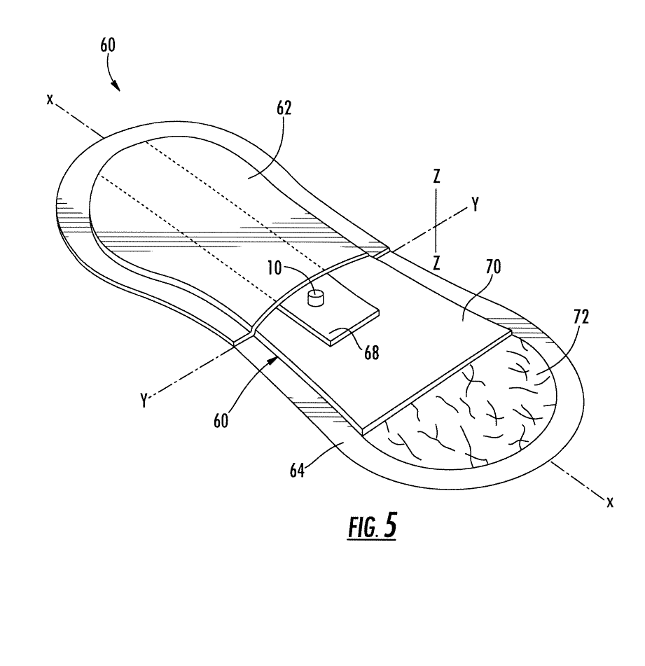

In another embodiment, the twisted, compressed substrate 10 can be included within a sanitary napkin 60 for feminine hygiene, as shown in FIG. 5. However, as discussed above, the twisted, compressed substrate 10 may be embodied in other types of feminine hygiene products. Nonetheless, in the illustrated embodiment, the sanitary napkin 60 includes a liner 62, a baffle 64, and an absorbent core 66, between any of which the twisted, compressed substrate 10 may be positioned. The absorbent core 66 is positioned inward from the outer periphery of the sanitary napkin 60 and includes a body-facing surface positioned adjacent the liner 62 and a garment-facing surface positioned adjacent the baffle 64.

Not only does the twisted, compressed substrate 10 indicate to the wearer that it has been insulted, but also the twisted, compressed substrate 10 helps maintain a close, secure fit of the sanitary napkin 60 with the body of the wearer.

The liner 62 is generally designed to contact the body of the user and is liquid-permeable. The liner 62 can surround the absorbent core 66 so that it completely encases the sanitary napkin 60. Alternatively, the liner 62 and the baffle 64 can extend beyond the absorbent core 66 and be peripherally joined together, either entirely or partially, using known techniques. Typically, the liner 62 and the baffle 64 are joined by adhesive bonding, ultrasonic bonding, or any other suitable joining method known in the art.

The liquid-permeable liner 62 is sanitary, clean in appearance, and somewhat opaque to hide bodily discharges collected in and absorbed by the absorbent core 66. The liner 62 further exhibits good strike-through and rewet characteristics permitting bodily discharges to rapidly penetrate through the liner 62 to the absorbent core 66, but not allow the body fluid to flow back through the liner 62 to the skin of the wearer. For example, some suitable materials that can be used for the liner 62 include nonwoven materials, perforated thermoplastic films, or combinations thereof. A nonwoven fabric made from polyester, polyethylene, polypropylene, bicomponent, nylon, rayon, or like fibers may be utilized. For instance, a white uniform spunbond material is particularly desirable because the color exhibits good masking properties to hide menses that has passed through it. U.S. Pat. Nos. 4,801,494 to Datta, et al. and 4,908,026 to Sukiennik. et al. teach various other cover materials that can be used in the present invention.

The liner 62 can also contain a plurality of apertures (not shown) formed therethrough to permit body fluid to pass more readily into the absorbent core 66. The apertures can be randomly or uniformly arranged throughout the liner 62, or they can be located only in the narrow longitudinal band or strip arranged along the longitudinal axis X-X of the sanitary napkin 60. The apertures permit rapid penetration of body fluid down into the absorbent core 66. The size, shape, diameter any number of apertures can be varied to suit one's particular needs.

As stated above, the absorbent article also includes a baffle 64. The baffle 14 is generally liquid-impermeable and designed to face the inner surface, i.e., the crotch portion of an undergarment (not shown). The baffle 64 can permit a passage of air or vapor out of the sanitary napkin 60, while still blocking the passage of liquids. Any liquid-impermeable material can generally be utilized to form the baffle 64. For example, one suitable material that can be utilized is a microembossed polymeric film, such as polyethylene or polypropylene. In particular embodiments, a polyethylene film is utilized that has a thickness in the range of about 0.2 mils to about 5.0 mils, and particularly between about 0.5 to about 3.0 mils.

As indicated above, the sanitary napkin 60 also contains an absorbent core 66 positioned between the liner 62 and the baffle 64. In the illustrated embodiment, for example, the absorbent core 66 contains three separate and distinct absorbent members 68, 70 and 72, between any of which the twisted, compressed substrate 10 may be positioned. It should be understood, however, that any number of absorbent members can be utilized in the present invention. For example, in one embodiment, only the absorbent member 72 may be utilized.

As shown, the first absorbent member 68, or intake member, is positioned between the liner 62 and the second absorbent member 70, or transfer delay member. The intake member 68 represents a significant absorbing portion of the sanitary napkin 60 and has the capability of absorbing at least about 80%, particularly about 90%, and more particularly about 95% of the body fluid deposited onto the sanitary napkin 60. In terms of amount of body fluid, the intake member 68 can absorb at least about 20 grams, particularly about 25 grams, and more particularly, about 30 or more grams of body fluid.

The intake member 68 can generally have any shape and/or size desired. For example, in one embodiment, the intake member 68 has a rectangular shape, with a length equal to or less than the overall length of the sanitary napkin 60, and a width less than the width of the sanitary napkin 60. For example, a length of between about 150 mm to about 300 mm and a width of between about 10 mm to about 40 mm can be utilized.

Typically, the intake member 68 is made of a material that is capable of rapidly transferring, in the z-direction, body fluid that is delivered to the liner 62. Because the intake member 68 is generally of a dimension narrower than the sanitary napkin 60, the sides of the intake member 68 are spaced away from the longitudinal sides of the absorbent article 60 and the body fluid is restricted to the area within the periphery of the intake member 68 before it passes down and is absorbed into the transfer delay member 70. This design enables the body fluid to be combined in the central area of the sanitary napkin 60 and to be wicked downward.

In general, any of a variety of different materials are capable of being used for the intake member 68 to accomplish the above-mentioned functions. For example, airlaid cellulosic tissues may be suitable for use in the intake member 68. The airlaid cellulosic tissue can have a basis weight ranging from about 10 grams per square meter (gsm) to about 300 gsm, and in some embodiments, between about 100 gsm to about 250 gsm. In one embodiment, the airlaid cellulosic tissue has a basis weight of about 200 gsm. The airlaid tissue can be formed from hardwood and/or softwood fibers. The airlaid tissue has a fine pore structure and provides an excellent wicking capacity, especially for menses.

A second absorbent member 70, or transfer delay member, is also positioned vertically below the intake member 68. In some embodiments, the transfer delay member 70 contains a material that is less hydrophilic than the other absorbent members, and may generally be characterized as being substantially hydrophobic. For example, the transfer delay member 70 may be a nonwoven fibrous web composed of a relatively hydrophobic material, such as polypropylene, polyethylene, polyester or the like, and also may be composed of a blend of such materials. One example of a material suitable for the transfer delay member 70 is a spunbond web composed of polypropylene, multi-lobal fibers. Further examples of suitable transfer delay member materials include spunbond webs composed of polypropylene fibers, which may be round, tri-lobal or poly-lobal in cross-sectional shape and which may be hollow or solid in structure. Typically the webs are bonded, such as by thermal bonding, over about 3% to about 30% of the web area. Other examples of suitable materials that may be used for the transfer delay member 70 are described in U.S. Pat. Nos. 4,798,603 to Meyer, et al. and 5,248,309 to Serbiak, et al., which are incorporated herein in their entirety by reference thereto for all purposes. To adjust the performance of the invention, the transfer delay member 70 may also be treated with a selected amount of surfactant to increase its initial wettability.

The transfer delay member 70 can generally have any size, such as a length of about 150 mm to about 300 mm. Typically, the length of the transfer delay member 70 is approximately equal to the length of the sanitary napkin 60. The transfer delay member 70 can also be equal in width to the intake member 68, but is typically wider. For example, the width of the transfer delay member 70 can be from between about 50 mm to about 75 mm, and particularly about 48 mm.

The transfer delay member 70 of the absorbent core 66 typically has a basis weight less than that of the other absorbent members. For example, the basis weight of the transfer delay member 20 is typically less than about 150 grams per square meter (gsm), and in some embodiments, between about 10 gsm to about 100 gsm. In one particular embodiment, the transfer delay member 70 is formed from a spunbonded web having a basis weight of about 30 gsm.

Besides the above-mentioned members, the absorbent core 66 also includes a composite member 72. For example, the composite member 72 can be a coform material. In this instance, fluids can be wicked from the transfer delay member 70 into the absorbent member 72. The composite absorbent member 72 may be formed separately from the intake member 68 and/or transfer delay member 70, or can be formed simultaneously therewith. In one embodiment, for example, the composite absorbent member 72 can be formed on the transfer delay member 70 or intake member 68, which acts a carrier during the coform process described above.

The sanitary napkin 60 may also contain other components as well. For instance, in some embodiments, the lower surface of the baffle 64 can contain an adhesive for securing the sanitary napkin 60 to an undergarment. In such instances, a backing (not shown) may be utilized to protect the adhesive side of the sanitary napkin 60 so that the adhesive remains clean prior to attachment to undergarment. The backing can generally have any desired shape or dimension. For instance, the backing can have a rectangular shape with dimension about 17 to about 21 cm in length and about 6.5 to 10.5 cm in width. The backing is designed to serve as a releasable peel strip to be removed by the user prior to attachment of the sanitary napkin 60 to the undergarment. The backing serving as a releasable peel strip can be a white Kraft paper that is coated on one side so that it can be released readily from the adhesive side of the sanitary napkin 60. The coating can be a silicone coating, such as a silicone polymer commercially available from Akrosil of Menasha, Wis.

Once formed, the sanitary napkin 60 generally functions to absorb and retain fluids, such as menses, blood, urine, and other excrements discharged by the body during a menstrual period. For example, the intake member 68 can allow the body fluid to be wicked downward in the z-direction and away from the liner 62 so that the liner 62 retains a dry and comfortable feel to the user. Moreover, the intake member 68 can also absorb a significant amount of the fluid. The transfer delay member 70 initially accepts fluid from the intake member 68 and then wicks the fluid along its length and width (−x and −y axis) before releasing the fluid to the composite absorbent member 72. The composite absorbent member 72 then wicks the fluid along its length and width (−x and −y axis) utilizing a greater extent of the absorbent capacity than the transfer delay member 70. Therefore, the composite absorbent member 72 can become completely saturated before the fluid is taken up by the transfer delay member 70. The fluid is also wicked along the length of the transfer delay member 70 and the composite absorbent member 72, thereby keeping the fluid away from the edges of the sanitary napkin 60. This allows for a greater utilization of the absorbent core 66 and helps reduce the likelihood of side leakage.

What is claimed:

1. An absorbent article configured to alert a wearer of an insult of the absorbent article, the absorbent article comprising:
a liquid-permeable layer;
a liquid-impermeable layer;
an absorbent core positioned between the liquid-permeable layer and the liquid-impermeable layer; and
a twisted, compressed substrate positioned between the liquid-permeable layer and the liquid-impermeable layer, wherein the twisted, compressed substrate defines a x-direction, a y-direction, and a z-direction, wherein the twisted, compressed substrate is configured to expand in the z-direction upon contact with a liquid to form an expanded substrate without substantially expanding in either the x-direction or the y-direction, and wherein the twisted, compressed substrate is configured to unwind upon contact with a liquid.

2. An absorbent article as in claim 1, wherein the twisted, compressed substrate unwinds at least about 360° upon contact with a liquid.

3. An absorbent article as in claim 1, wherein the twisted, compressed substrate unwinds at least about 720° upon contact with a liquid.

4. An absorbent article as in claim 1, wherein the twisted, compressed substrate unwinds about 1080° to about 1800° upon contact with a liquid.

5. An absorbent article as in claim 1, wherein the twisted, compressed substrate is configured to at least double in size in the z-direction upon contact with a liquid.

6. An absorbent article as in claim 1, wherein the twisted, compressed substrate is configured to at least triple in size in the z-direction upon contact with a liquid.

7. An absorbent article as in claim 1, wherein the twisted, compressed substrate is configured to expand from about 5 times to about 10 times of its size in the z-direction upon contact with a liquid.

8. An absorbent article as in claim 1, wherein the twisted, compressed substrate is configured to expand only up to about 110% of its original size in both the x-direction and the y-direction.

9. An absorbent article as in claim 1, wherein the twisted, compressed substrate is configured to expand only from about 100.5% to about 105% of its original size in both the x-direction and the y-direction.

10. An absorbent article as in claim 1, wherein the compression molded web comprises a nonwoven web of pulp staple fibers.

11. An absorbent article as in claim 1, wherein the compression molded web comprises fibers formed from a synthetic polymer.

12. An absorbent article as in claim 1, wherein the twisted, compressed substrate has a cylindrical shape.

13. An absorbent article as in claim 1, wherein the twisted, compressed substrate is positioned between the absorbent core and the liquid-permeable layer.

14. An absorbent article as in claim 1, wherein the twisted, compressed substrate is positioned between the absorbent core and the liquid-impermeable layer.

15. A method of making a twisted, compressed substrate, the method comprising:
twisting a web material into a twisted, cylindrical tube;
positioning the twisted, cylindrical tube into an elongated barrel;
subjecting the twisted, cylindrical tube to a compression force in a direction of the elongation of the barrel, wherein the compression force is provided by moving a pressing rod through the elongated barrel.

16. A method as in claim 15 wherein the step of twisting a web material into the twisted, cylindrical tube comprises:
wetting the web material;
rolling the web material into a cylindrical tube;
twisting the web material into the twisted, cylindrical tube;
thereafter, drying the twisted, cylindrical tube.

17. A method as in claim 15, wherein the twisted, cylindrical tube is twisted such that one end of the twisted, cylindrical tube rotates at least about 360° relative to an opposite end of the twisted, cylindrical tube.

18. A method as in claim 15, wherein the twisted, cylindrical tube is twisted such that one end of the twisted, cylindrical tube rotates at least about 720° relative to an opposite end of the twisted, cylindrical tube.

19. A method as in claim 15, wherein the twisted, cylindrical tube is twisted such that one end of the twisted, cylindrical tube rotates from about 1080° to about 1800° relative to an opposite end of the twisted, cylindrical tube.

20. A method as in claim 15 further comprising
positioning the twisted, compressed substrate between a liquid-permeable layer and a liquid impermeable layer of an absorbent article.
